(19) United States
(12) Patent Application Publication
(10) Pub. No.: US 2020/0008703 A1
(43) Pub. Date: Jan. 9, 2020

(54) METHOD FOR MONITORING A PATIENT DURING A MEDICAL IMAGING EXAMINATION

(71) Applicant: Siemens Healthcare GmbH, Erlangen (DE)

(72) Inventor: Mario Zeller, Erlangen (DE)

(73) Assignee: Siemens Healthcare GmbH, Erlangen (DE)

(21) Appl. No.: 16/458,659

(22) Filed: Jul. 1, 2019

(30) Foreign Application Priority Data
Jul. 4, 2018 (DE) 10 2018 210 973.7

(51) Int. Cl. A61B 5/055; A61B 5/113; A61B 6/03; A61B 5/00

(52) U.S. Cl. CPC A61B 5/055; A61B 5/113; A61B 6/04; A61B 5/7285; A61B 5/742; A61B 6/032

(57) ABSTRACT

In a method for monitoring a patient during a medical imaging examination, at least one state signal of the patient is detected using at least one state detector, and the at least one detected state signal of the patient is evaluated using an evaluation algorithm. An emotional state of the patient can be established based on the detected state signal. The method can further include generating output information based on the established emotional state of the patient and outputting the output information via an output interface.

### CROSS REFERENCE TO RELATED APPLICATIONS

This patent application claims priority to German Patent Application No. 102018210973.7, filed Jul. 4, 2018, which is incorporated herein by reference in its entirety.

### BACKGROUND

### Field

The present disclosure relates to a method for monitoring a patient during a medical imaging examination, in particular a magnetic resonance examination. The present disclosure furthermore relates to a medical imaging apparatus that is configured to monitor a patient during a medical imaging examination, including performing the method according to aspects described herein, as well as to a corresponding computer program product and an electronically-readable data medium with a corresponding computer program.

### Related Art

Medical imaging examinations, in particular magnetic resonance examinations, frequently represent a major challenge for patients, since the patient is to lie as still as possible during the long examination time and is to make as few movements as possible or no movements at all, since movements can have a negative influence of the image quality of the image data acquired. These demands made on the patients can lead however to patients feeling unwell and/or stressed during the medical imaging examination. In such a state, patients often make involuntary movements, which in their turn can have a negative effect on the image quality of the acquired image data of medical imaging examination. Another consequence of this can be a premature aborting of the medical imaging examination.

Also it has previously often been difficult for medical staff to correctly estimate an emotional state, and/or a mood of the patient during the duration of the medical imaging examination, in order to have a calming influence on the patient. Even if medical staff have a clear sight of the patient during the medical imaging examination, the positioning of the patient within a tunnel-shaped and/or cylindrical patient receiving area of the medical imaging apparatus, such as for example a magnetic resonance apparatus, does not allow the medical staff to recognize a facial expression and/or the look on the patient's face, in order to divine the mood of the patient therefrom.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a schematic illustration of a medical imaging apparatus according to an exemplary embodiment of the present disclosure.

FIG. 2 is a flowchart of a monitoring method according to an exemplary embodiment of the present disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure.

An object of the present disclosure is to support medical staff in looking after the patient during a medical imaging examination.

The disclosure includes a method for monitoring a patient during a medical imaging examination, such as a magnetic resonance examination. In an exemplary embodiment, the method includes: detecting at least one state signal of the patient by at least one state detector, evaluating the at least one detected state signal of the patient using an evaluation algorithm of a processor, where the evaluation algorithm establishes an emotional state of the patient on the basis of the detected state signal, and generating output information based on the established emotional state of the patient and outputting the output information by an output.

The medical imaging examination can include all the medical imaging examinations as would be understood by one of ordinary skill in the art, such as, for example, a computed tomography examination, a PET (Positron Emission Tomography) examination, or a magnetic resonance examination. Magnetic resonance examinations have a relatively long measurement time of up to one hour and more, during which the patient should remain as calm and motionless as possible. Moreover, a patient receiving area of a magnetic resonance apparatus also has a particularly narrow and tunnel-shaped design, so that patients can often experience states of claustrophobic anxiety here during a magnetic resonance examination. This being the case, the method for monitoring a patient during a medical imaging examination according to exemplary embodiments can be used to particular advantage during magnetic resonance examinations.

In an exemplary embodiment, the at least one state signal of the patient includes a signal that describes a state of the patient, which can include, for example, a movement state of the patient. In an exemplary embodiment, the state of the patient additionally or alternative includes a heart cycle of the patient, a pulse signal of the patient, a breathing cycle of the patient, and/or a facial expression and/or a look on the patient's face.

In an exemplary embodiment, if a number of different state signals of the patient are detected, a separate state detector is provided for each of the different state signals for the detection of the respective state signal. The state detector or also the number of state detectors can already be integrated into the medical imaging apparatus, in particular into a scanner of a magnetic resonance apparatus, or can be arranged in the scanner. For example, state detectors that each comprise a camera for detecting a movement of the patient can already be arranged and/or integrated into a housing of a patient receiving area of the scanner. In an exemplary embodiment, the camera is configured to detect a movement state of the patient and/or a facial expression of the patient during the medical imaging examination.

In an exemplary embodiment, the at least one state detector is integrated with a radio-frequency antenna. Here the state detector is configured to send out and/or detect a pilot tone signal. In an exemplary embodiment, the state detector includes the radio-frequency antenna and a pilot tone transmitter and/or a pilot tone sensor integrated within the radio-frequency antenna. In an exemplary embodiment, a state detector embodied in this way is configured to detect both a movement state of the patient and also a heart cycle of the patient and/or a breathing cycle of the patient during the medical imaging examination.

In an exemplary embodiment, the state detector includes additional detectors or sensors that are additionally arranged and/or positioned on the patient for the medical imaging examination, in particular for the magnetic resonance examination. For example, the state detector can include an EKG unit and/or a breathing sensor and/or a pulse sensor, in particular a pulse oximeter, so that, a state detector embodied in such a way can detect a breathing signal and/or breathing cycle of the patient and/or a pulse signal of the patient and/or a heart cycle of the patient during the medical imaging examination.

In an exemplary embodiment, the evaluation of the detected state signals of the patient is performed by a processor, which has an evaluation algorithm configured to perform the evaluation. Here an emotional state of the patient is determined and/or established by the processor, in particular by the evaluation algorithm, on the basis of the detected state signals. In particular the evaluation algorithm uses the detected state signals as input parameters and/or input values for computing the emotional state of the patient. For establishing and/or determining the emotional state of the patient the detected state signals are evaluated for example in respect of a predetermined critical threshold value and/or in respect of a change over a period of time and/or in respect of characteristic features.

The inventive processor includes at least one processing module and/or a processor, wherein the processor is embodied for carrying out a method. Thus the processor is embodied in particular for executing computer-readable instructions in order to carry out the inventive method. In particular the processor includes a memory, wherein computer-readable information is stored on the memory, wherein the processor is embodied to load the computer-readable information from the memory and to execute the computer-readable information in order to carry out an inventive method. In this way the inventive processor is embodied for carrying out a method for monitoring a patient during a medical imaging examination, in particular a magnetic resonance examination.

The components of the processor can be embodied predominantly in the form of software components. Basically however these components, in particular when it is a matter of especially fast computations, can also be realized partly in the form of software-supported hardware components, for example FPGAs or the like. Likewise the interfaces needed, for example when it is only a matter of transferring data from other software components, can be embodied as software interfaces. They can however also be embodied as interfaces constructed from hardware, which are activated by suitable software. Of course it is also conceivable for a number of the said components to be realized in a group in the form of an individual software component or software-supported hardware components.

The medical imaging apparatus can include the processor. The processor can also be installed separately from the medical imaging apparatus. The processor can also be connected to the medical imaging apparatus. The processor can be embodied to send control signals to the medical imaging apparatus and/or to receive and/or to process control signals. The processor can be integrated into the medical imaging apparatus.

An emotional state of the patient is to be understood in particular as the condition of the patient and/or a disposition of the patient during the medical imaging examination. The emotional state of the patient in this case can include various categories of emotional states. If the patient is in a calm emotional state for example, no adverse effect on the medical imaging examination is to be expected. If on the other hand the patient is in a nervous emotional state and/or stressed emotional state, there are likely to be movements of the patient that would result in a deterioration of the image quality of the image data acquired.

In an exemplary embodiment, the processor is configured to generate the output information based on the established and/or determined emotional state of the patient. Subsequently, the generated output information can be provided by the processor for an output to medical staff and/or transferred to an output. The output information can be, for example, a text output, images, videos, a symbol, and/or a number of symbols that characterize the current emotional state and/or emotional states of the patient established, such as, for example an emoji and/or smiley.

In an exemplary embodiment, the output is arranged within the room within which the medical operating staff are monitoring the medical imaging examination. In an exemplary embodiment, the output includes a display, a monitor, and/or one or more other output interfaces or devices as would be understood by one of ordinary skill in the relevant arts.

In an exemplary embodiment, the method advantageously enables the medical operating staff to be supported while looking after the patient during the medical imaging examination. In particular, the medical operating staff can be kept informed by the output of the output information about the current emotional state of the patient. Moreover in this way the medical operating staff can take timely action to calm the patient as soon as the latter starts to become nervous during the medical imaging examination. In this way the likelihood of a patient-initiated abortion of the measurement during the medical imaging examination and thus also of a medical imaging examination having to be repeated because of a patient-initiated abortion can be reduced.

In an exemplary embodiment, a provision for the state signal of the patient includes a signal of a movement of the patient and/or a signal of a detected facial expression of the patient and/or a signal of a heart cycle of the patient and/or a breathing signal of the patient and/or a pulse signal of the patient. Such state signals are especially sensitive to a change of an emotional state of the patient. Consequently, an evaluation algorithm is configured to determine/establish a current emotional state of the patient with high reliability. If the emotional state of the patient goes from a calm emotional state to a nervous emotional state, this can be recognized for example by an increased movement of the patient and/or by an increased breathing frequency and/or a faster heart cycle etc. in the state data.

In an exemplary embodiment, a single state detector is provided that is configured to detect only state signals of the same category are always available for evaluation and/or the determination of the emotional state of the patient. In an exemplary embodiment, two or more state detectors can also be available, so that state signals of different categories are available for the determination of the emotional state of the patient.

It can be determined from the detected movements of the patient, for example, whether the patient is currently in a calm phase, i.e. a phase with few or no movements, or in a restless phase, i.e. in a phase in which the patient is making many movements. A restless phase can be seen here as an indication of a nervous and/or stressed state of the patient. From the facial expression of the patient detected it can be established for example whether the patient has relaxed facial features, which point to a calm phase of the patient. Moreover it can also be concluded from the facial expression detected, in particular from an eye area and a mouth area of the patient, that the patient is in a stress situation and/or pain situation. For example, such a situation can be ascertained from a restless movement of the eyes and/or a furrowed brow and/or narrowed eyes and/or distorted angles of the mouth etc.

When the patient is in a calm emotional state, the signal of the heart cycle of the patient should include a heart cycle that is as even as possible. On the basis of a heart cycle, it can be established, for example, whether the patient is in a calm phase or in a restless and/or stressed phase. The frequency of the heart cycle can be compared here with a critical threshold value, wherein the critical threshold value can preferably be specifically tailored to the patient.

The breathing signal of the patient includes a breathing signal that is as even as possible when the patient is in a calm emotional state. It can be established on the basis of a breathing frequency for example whether the patient is in a calm phase or in a restless and/or stressed phase. The breathing frequency can be compared here with a critical threshold value, wherein the critical threshold value can preferably be specifically tailored to the patient.

The pulse signal of the patient includes a pulse signal that is as even as possible when the patient is in a calm emotional state. It can be established on the basis of a pulse frequency for example whether the patient is in a calm phase or in a restless and/or stressed phase. The pulse frequency can be compared here with a critical threshold value, wherein the critical threshold value can preferably be specifically tailored to the patient.

In an exemplary embodiment, the detection of the at least one state signal of the patient is carried out continuously during the medical imaging examination. Through this, a current emotional state of the patient during the duration of the medical imaging examination is provided at all times for the medical operating staff looking after the medical imaging examination. In this way the medical operating staff can recognize a critical situation of the patient during the medical imaging examination at any time and, where required, can calm the patient down. Preferably the detection of the at least one state signal of the patient is done continuously during the entire duration of the medical imaging examination.

In an exemplary embodiment, at least two or more different state signals for the determination and/or establishment of the emotional state of the patient are detected and evaluated. The two or more different state signals can be assigned to different state categories, such as for example, a state category of a movement of the patient, state category of a facial expression of the patient, a state category of a heart cycle of the patient, a state category of a breathing signal of the patient and/or a state category of a pulse signal of the patient etc. This embodiment of the disclosure makes possible a high reliability in the determination of a current emotional state of the patient. In particular, through this embodiment, the current emotional state of the patient can be detected and/or determined redundantly.

In an exemplary embodiment, the evaluation algorithm is configured to determine a change of an emotional state of the patient based on at least one change of a detected state signal. For example, the detection of an increase in a movement of the patient and/or the detection of an increase in a breathing frequency and/or the detection in an increase of a heart frequency etc. enables it to be deduced that the patient is becoming increasingly agitated and/or that there is an increasingly agitated phase of the patient. In an exemplary embodiment, the detection of a decrease in a movement of the patient and/or the detection of a decrease in a breathing frequency and/or the detection in a decrease of a heart frequency etc. enables it to be concluded that the patient is becoming increasingly calmer and/or that there is an increasingly calm phase of the patient. In particular in this way tendencies of a transition of the patient from a calm phase into a nervous phase or conversely from a nervous phase into a calm phase can already be detected and established and shown to the medical operating staff. This also makes it possible for the medical operating staff to intervene at an early stage with the patient at the beginning of a nervous phase, so that a rapid calming of the patient can occur and/or can be instigated.

In an exemplary embodiment, the evaluation algorithm is configured to determine the change in the detected state based on a comparison of the detected state signal with a predetermined critical threshold value. In this way it can be determined especially easily at what point a critical state, in particular a critical emotional state, of the patient is reached, which requires an intervention of the medical operating staff. The predetermined critical threshold value can preferably be defined in this case specifically for the patient. Thus for example a critical threshold value for the pulse signal and/or a heart frequency can be dependent on a resting pulse and a heart frequency of a calm phase of the patient. In particular a patient can be individually monitored and/or looked after during the medical imaging examination in this way.

In an exemplary embodiment, the evaluation algorithm is configured to predetermine, when evaluating two or more different state signals, that, for a determination and/or establishment of a critical emotional state of the patient, at least 50% of the different state signals are greater than a predetermined critical threshold value. In an exemplary embodiment, the two or more different state signals of the patient can each be assigned to a different state category. In an exemplary embodiment, at least 55% of the different state signals, in particular the different state signals from the two or more different state categories, fulfill the conditions for a determination of a critical emotional state of the patient. In an exemplary embodiment, at least 60% of the different state signals, in particular the different state signals from the two or more different state categories, fulfill the conditions for a determination of a critical emotional state of the patient. In an exemplary embodiment, at least 65% of the different state signals, in particular the different state signals from the two or more different state categories, fulfill the conditions for a determination of a critical emotional state of the patient. In an exemplary embodiment, at least 70% of the different state signals, in particular the different state signals from the two or more different state categories, fulfill the conditions for a determination of a critical emotional state of the patient. This enables an advantageously high reliability to be achieved in the determination and/or establishment of the current emotional state of the patient.

In an exemplary embodiment, the evaluation algorithm is configured to determine the emotional state of the patient using a trained artificial neural network.

In this way, the evaluation of the detected state signals is based on a machine learning method, also called a deep-learning method, which is based on the artificial neural network. An artificial neural network (ANN) is in particular a network of artificial neurons emulated in a computer program. The artificial neural network is typically based in this case on a networking of a number of artificial neurons. The artificial neurons in this case are typically arranged in different layers. Usually the artificial neural network includes an input layer and an output layer, of which the neuron output is visible as the only layer of the artificial neural network. Layers lying between the input layer and the output layer are typically referred to as hidden layers. Typically an architecture and/or topology of an artificial neural network is first of all initiated and then in a training phase trained for a specific task or for a number of tasks. The training of the artificial neural network in this case includes an alteration of a weighting of a connection between two artificial neurons of the artificial neural network. The training of the artificial neural network can also include a development of new connections between artificial neurons, a deletion of existing connections between artificial neurons, an adaptation of threshold values of the artificial neurons and/or an insertion or a deletion of artificial neurons.

In an exemplary embodiment, the artificial neural network has already been suitably trained beforehand for the evaluation of the detected state data. Training image datasets have been used in particular in this case for the training of the artificial neural network, in which for example an emotional state is already assigned to a detected facial expression of a patient. The medical training datasets in this case have typically been acquired from patients of different training persons and/or training patients.

In an exemplary embodiment, taking account of the detected state signals, for example, a detected facial expression and/or a detected look on the patient's face, enables the determination and/or establishment of the emotional state of the patient to be tailored specifically to the patient using the artificial neural network. It is also conceivable for the trained artificial neural network to be suitably adapted retroactively on the basis of the detected state signal, in particular a detected facial expression and/or a detected look on the patient's face, so that the artificial neural network can carry out the determination and/or establishment of the emotional state of the patient especially advantageously tailored to the detected facial expression and/or detected look on the patient's face. For example it is conceivable for the artificial neural network to take over the emotional state of the patient for a detected facial expression and/or detected look on the patient's face. It is also conceivable for the detected facial expression and/or the detected look on the patient's face or information derived therefrom to be taken into account as additional training parameters during training of the artificial neural network.

In an exemplary embodiment, the neural network used for the evaluation is configured to assign or associate a detected facial expression to an emotional state of the patient. This has the advantage that this type of complex process is able to be carried especially quickly and effectively using the artificial neural network.

In an exemplary embodiment, the output information is output continuously to a user, in particular to a medical operator. In an exemplary embodiment, the output information is output to the user during the entire duration of the medical imaging examination. This makes possible a permanent monitoring of the patient during the medical imaging examination, in particular during the entire duration of the medical imaging examination, so that a current emotional state of the patient is communicated at all times to the operating staff using the output information. This enables a critical patient situation to be recognized during the entire duration of the medical imaging examination and such a situation of the patient to be counteracted by the medical staff. An abort rate of medical imaging examinations as a result of the patient being unwell and/or a critical situation of the patient can advantageously be reduced.

In an exemplary embodiment, the processor is configure to automatically establish and provide a suggestion for adapting a measurement strategy and/or an examination strategy of the medical imaging examination based on the established emotional state of the patient. This makes possible an advantageous supporting of the medical operating staff during the medical imaging examination. In particular in this way a suggestion can be made to the medical operating staff, which provides for a use of fast measurement sequences and/or a rearrangement of the course of the measurement, so that important image data is acquired first, and/or dispensing with a planned administration of contrast media etc. In an exemplary embodiment, the suggestion for adapting the measurement strategy and/or an examination strategy is output to the user by the output. In this case there can be provision for the user merely to have to agree to such a suggestion and for the implementation of the suggestion to be carried out automatically and/or autonomously by the processor.

In an exemplary embodiment of the disclosure, a medical imaging apparatus, in particular a magnetic resonance apparatus is provided. In an exemplary embodiment, the medical imaging apparatus includes scanner, a patient receiving area surrounded by the scanner, at least one state detector, a processor and a user interface with an output. In this example, the medical imaging apparatus is configured to carry out a method for monitoring a patient during a medical imaging examination, in particular a magnetic resonance examination, according to one or more embodiments described herein.

In an exemplary embodiment, the medical imaging apparatus is any medical imaging apparatuses as would be understood by one of ordinary skill in the art, such as, for example, a computed tomography apparatus, a PET apparatus (Positron Emission Tomography apparatus), or a magnetic resonance apparatus. Cylinder-shaped and/or tunnel-shaped patient receiving areas of a magnetic resonance apparatus, may result in the patients feeling hemmed while in the patient receiving area. Depending on the patient, this can lead to states of anxiety, which can lead to a magnetic resonance examination being aborted prematurely. The method according to exemplary embodiments advantageously reduces and/or presents premature aborting of the measurement of a patient. In particular, the medical operating staff can be supported in this way when looking after the patient during the medical imaging examination. In particular the medical operating staff can be kept informed by the output of the output information about a current emotional state of the patient. Moreover in this way the medical operating staff can make timely interventions to calm down the patient as soon as said patient starts to become nervous during the medical imaging examination. In this way a likelihood of a patient-initiated aborting of the medical imaging examination can be reduced and thus also the likelihood that a medical imaging examination has to be repeated as a result of a patient-initiated aborting.

The medical imaging apparatus according to exemplary embodiments is configured to perform the operations of the method for monitoring a patient during a medical imaging examination according to one more aspects, and thereby can realize the advantages of the method described herein. Features, advantages or alternate forms of aspects of the method can likewise be transferred to the other claimed subject matter (e.g. apparatus) and vice versa.

In an exemplary embodiment, a magnetic resonance apparatus (as the medical imaging apparatus), the scanner includes a magnet unit with a super-conducting main magnet, a gradient coil and a radio-frequency antenna. The state detector in this case can already be integrated within the scanner, for example as a camera arranged within the patient receiving area, whereby an especially compact medical imaging apparatus for carrying out the inventive method can be provided. As an alternative or in addition the state detector can also be embodied separately to the scanner.

In an exemplary embodiment, the medical imaging apparatus includes at least one state detector having a camera, breathing sensor, pulse detector, heart signal detector, and/or one or more other detectors or sensors as would be understood by one of ordinary skill in the art. In an exemplary embodiment, the camera detects a movement state of the patient and/or a facial expression of a patient during the medical imaging examination. Moreover the at least one state detector can be integrated within a radio-frequency antenna. Here the state detector can be embodied for sending out and/or detection of a pilot-tone signal, wherein the state detector can include the radio-frequency antenna as well as just a pilot-tone transmitter and/or a pilot-tone sensor integrated within the radio-frequency antenna. Both a movement state of the patient and also a heart cycle of the patient and/or a breathing cycle of the patient during the medical imaging examination can be detected by a state detector embodied in this way.

In an exemplary embodiment, as well as a pilot-tone sensor, the heart signal detector can also include an electrocardiogram (EKG) unit. The pulse detector can include a pulse oximeter. The breathing sensor can likewise include a pilot-tone sensor or also a respiratory belt etc.

In an exemplary embodiment, the computer program product is able to be loaded directly into a memory of a programmable processor and has program code configured to carry out a method according to one or more embodiments when the computer program product is executed in the processor. The computer program product can be a computer program or can include a computer program. This enables a method according exemplary embodiments to be carried out in a manner which is quick, identically repeatable and robust. The computer program product is configured in such a way that in can carry out the method operations by the processor. In an exemplary embodiment, the processor includes an appropriate main memory, an appropriate graphics card or an appropriate logic circuit, so that the respective method operations can be carried out efficiently by the processor. The computer program product is stored in a computer-readable medium, for example, and/or is held on a network or a server, from where it can be loaded into the processor, which can be directly connected thereto. Furthermore control information of the computer program product can be stored on an electronically-readable data medium. The control information of the electronically-readable data medium can be designed in such a way that, when the data medium is used in a processor, it carries out an inventive method. Thus the computer program product can also represent the electronically-readable data medium. Examples of electronically-readable data media are a DVD, a magnetic tape, a hard disk, memory, a solid-state drive, a USB stick, or other medium as would be understood by one of ordinary skill in the art on which electronically-readable control information, in particular software (cf. above) is stored. If this control information (e.g. software) is read from the data medium and stored in a controller and/or processor, all inventive forms of embodiment of the methods described previously can be carried out. Thus the disclosure can also be based on the computer-readable medium and/or the electronically-readable data medium.

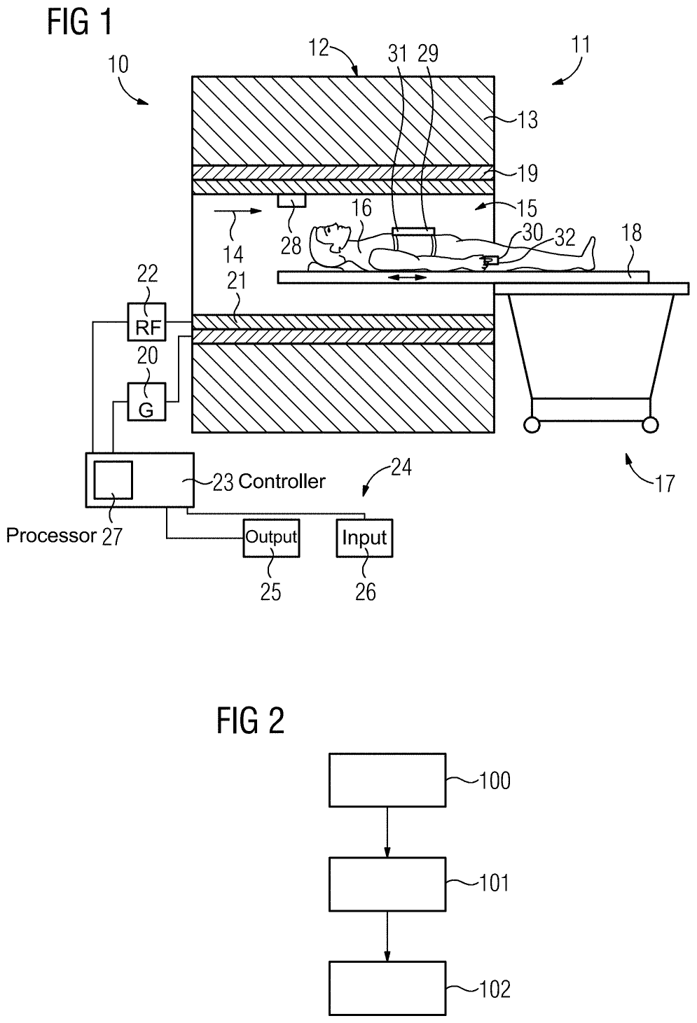

Shown schematically in FIG. 1 is a medical imaging apparatus 10. In an exemplary embodiment, the medical imaging apparatus 10 is formed by a magnetic resonance apparatus 11. The present disclosure will be explained with reference to the magnetic resonance apparatus 11 by way of example, but is not limited to the imaging methodology. The present disclosure is also applicable to, for example, computed tomography and/or PET, or one or more other medical imaging technologies as would be understood by one of ordinary skill in the relevant arts.

In an exemplary embodiment, the medical imaging apparatus 10 (the magnetic resonance apparatus 11) includes a scanner 12, which in the present exemplary embodiment is formed by a magnet unit of the magnetic resonance apparatus 11. In an exemplary embodiment, the scanner 12, in particular the magnet unit, includes a superconducting main magnet 13 for generating a strong and constant main magnetic field 14. Moreover, the scanner 12 has a patient receiving area 15 of the medical imaging apparatus 10, in particular of the magnetic resonance apparatus 11, for receiving a patient 16. The patient receiving area 15 in the present exemplary embodiment is embodied in a cylindrical shape and is surrounded in a cylindrical shape in a circumferential direction by the scanner 12. However, the patient receiving area 15 of a different shape/configuration is always conceivable. The patient 16 can be pushed and/or driven by a patient support apparatus 17 of the medical imaging apparatus 10, in particular of the magnetic resonance apparatus 11, into the patient receiving area 15. To this end the patient support apparatus 17 has a patient table 18 designed so that it can move within the patient receiving area 15.

In an exemplary embodiment, the scanner 12, in particular the magnet unit, furthermore has a gradient coil 19 for generation of magnetic field gradients, which are used for a spatial encoding during imaging. The gradient coil 19 is controlled by a gradient controller 20 of the magnetic resonance apparatus 11. The scanner 12, in particular the magnet unit, furthermore includes a radio-frequency antenna 21 for generating a polarization, which arises in the main magnetic field 14 generated by the main magnet 13. The radio-frequency antenna 21 is controlled by a radio-frequency antenna controller 22 of the magnetic resonance apparatus 11 and radiates radio-frequency magnetic resonance sequences into an examination space, which is essentially formed by a patient receiving area 15 of the magnetic resonance apparatus 11.

In an exemplary embodiment, the medical imaging apparatus 10, such as the magnetic resonance apparatus 11, includes a controller 23 that is configured to control the main magnet 13, the gradient controller 20, and/or the radio-frequency antenna controller 22. In an exemplary embodiment, the controller 23 centrally controls the medical imaging apparatus 10, in particular the magnetic resonance apparatus 11, such as for example the carrying out of a predetermined imaging gradient echo sequence. Moreover the controller 23 includes an evaluator not shown in any greater detail for evaluating medical imaging data that is acquired during the medical imaging examination, in particular the magnetic resonance examination. In an exemplary embodiment, the controller 23, gradient controller 20, and/or radio-frequency antenna controller 22 include processor circuitry that is configured to perform one or more respective operations/functions of the controller 23, gradient controller 20, and/or radio-frequency antenna controller 22.

In an exemplary embodiment, the medical imaging apparatus 10, in particular the magnetic resonance apparatus 11, includes a user interface 24, which is connected to the controller 23. Control information, such as for example imaging parameters, as well as reconstructed magnetic resonance images can be displayed on an output 25, for example in at least one monitor, of the user interface 24 for medical operating staff. Furthermore the user interface 24 has an input 26, of which information and/or parameters can be entered by the medical operating staff during a measurement process.

The medical imaging apparatus 10 shown, in particular the magnetic resonance apparatus 11, can of course include further components as would be understood by one of ordinary skill in the art. A general way in which a medical imaging apparatus 10, in particular the magnetic resonance apparatus 11, functions is also known to the person skilled in the art, so that a more detailed description of the further components has been omitted for brevity.

In an exemplary embodiment, the medical imaging apparatus 10, in particular the magnetic resonance apparatus 11, moreover includes a processor 27 and at least one state detector 28, 29, 30. The processor 27 in the present exemplary embodiment is included in the controller 23. As an alternative or in addition the processor 27 can also be arranged separately from the controller 23 within the magnetic resonance apparatus 11.

Furthermore, in the present exemplary embodiment, the medical imaging apparatus 10, in particular the magnetic resonance apparatus 10, has a number of state detectors 28, 29, 30. A first state detector 28 is integrated within the scanner 12 and includes at least one camera. A second state detector 29 is integrated within a local radio-frequency antenna 31. The state detector 29 can be embodied here for sending out and/or detecting a pilot-tone signal, wherein the state detector 29 is included in the radio-frequency antenna 31. As an alternative or in addition, the state detector can also just include a pilot-tone transmitter and/or a pilot-tone sensor integrated within the radio-frequency antenna 29. Further state detectors 30 are integrated in accessory units 32 that are used for the medical imaging examination, in particular the magnetic resonance examination and/or are included by said units. For example the one state detector 30 can include a breathing sensor and/or an EKG unit and/or a pulse oximeter.

In an exemplary embodiment, the medical imaging apparatus 10, in particular the magnetic resonance apparatus 11, includes just a single state detector 28, 29, 30 or just a selection of the state detectors 28, 29, 30 enumerated above for carrying out an inventive method for monitoring a patient 26 during a medical imaging examination, in particular a magnetic resonance examination.

The processor 27, together with the at least one state detector 28, 29, 30 and the output 25 is configured to perform the method for monitoring a patient 16 during a medical imaging examination, in particular a magnetic resonance examination, according to one or more exemplary embodiments. To this end the processor 27 includes the required software and/or computer programs, which are stored in a memory of the processor 27 not shown in any greater detail. The software and/or computer programs include one or more programs, algorithms, logic, and/or codes configured to carry out the method according to one or more embodiments, when the computer program and/or the software is executed by the processor 27. The memory can be included in the processor 27 and/or embodied separately from the processor 27. In this case, the memory can be arranged within the controller 23 and/or the magnetic resonance apparatus 11 and/or can also include an external memory, where the processor 27 can access the memory via a data or communication network.

Shown schematically in FIG. 2 is the inventive method for monitoring a patient 16 during a medical imaging examination, in particular a magnetic resonance examination. In an exemplary embodiment, in a first method step 100 of the method, there is a detection of at least one state signal of the patient 16 using at least one state detector 28, 29, 30. In the present exemplary embodiment, a number of state signals of the patient 16 are detected using the number of state detectors 28, 29, 30, where the number of state signals include different state signals of the patient 16, which are included in different state categories. For example, the state signal of the patient 16 can include a signal of a movement of the patient 16, a detected facial expression of the patient 16, a signal of a heart cycle of the patient 16, a breathing signal of the patient 16, and/or a pulse signal of the patient 16. In such cases, the different state signals can be assigned to different state categories, such as for example, to a state category of a movement of the patient 16 and/or to a state category a facial expression of the patient 16 and/or to a state category a heart cycle of the patient 16 and/or to a state category of a breathing signal of the patient 16 and/or to a state category of a pulse signal of the patient 16 etc.

In an exemplary embodiment, the detection of the at least one state signal of the patient 16 is done continuously during the medical imaging examination. Particularly advantageously, the detection of the state signals of the patient 16 is done continuously during the entire medical imaging examination, in particular the entire magnetic resonance examination.

After the detection of the state signals of the patient 16, the detected state signals are transmitted by a data transmitter not shown in any greater detail from the individual state detectors 28, 29, 30 to the processor 27. The transmitter can be a wired and/or wireless transmitter.

In an exemplary embodiment, in a second method step 101, there is an evaluation of the at least one detected state signal of the patient 16 by an evaluation algorithm of the processor 27. The evaluation algorithm establishes an emotional state of the patient 16 based on the detected state signal of the patient 16. In an exemplary embodiment, all detected and/or available state signals of the patient 16 are used for establishing the emotional state of the patient 16, but a subset can be used on other aspects.

In an exemplary embodiment, to establish and/or determine the emotional state of the patient 16, the detected state signals are evaluated, for example, in respect of a predetermined critical threshold value and/or in respect of a change over a period of time and/or in respect of characteristic features of the evaluation algorithm of the processor 27. In an exemplary embodiment, the evaluation algorithm determines, for example, in such cases a change of an emotional state of the patient 16 on the basis of a change of one of the detected state signals. It can be established from the detected movements of the patient 16 for example whether the patient 16 is currently in a calm phase, i.e. a phase with few or no movements, or in a restless phase, i.e. in a phase in which the patient 16 is making many movements. A restless phase in such cases can be seen as an indication of a nervous and/or stressed state of the patient 16.

Moreover it can be established, for example, from detected facial expression of the patient 16, whether the patient 16 has relaxed facial features, in particular a relaxed eye area and a relaxed mouth area of the patient 16, which point to a calm phase of the patient 16. Moreover a stress situation and/or a pain situation of the patient 16 can also be deduced from the facial expression detected, in particular from an eye area and a mouth area of the patient 16. For example such a situation can be determined from a restless eye movement and/or a furrowed brow and/or reduced eye openings and/or distorted mouth angles etc. of the patient 16.

An increasingly restless phase of the patient 16 can also be deduced from an increasing movement of the patient 16 and/or from an increase in a breathing frequency of the patient 16 and/or an increase in a heart frequency of the patient 16 etc. Conversely an increasing calmness of the patient 16 can also be deduced from a decreasing movement of the patient 16 and/or from a decrease in a breathing frequency of the patient 16 and/or a decrease in a heart frequency of the patient 16 etc.

In an exemplary embodiment, a change in one of the detected state signals of the patient 16 can be determined in the second method step 101 by the evaluation algorithm in respect of a comparison of the detected state signals with a predetermined critical threshold value. In an exemplary embodiment, the predetermined critical threshold value is defined as a patient-specific value. For example, a critical threshold value for the pulse signal and/or a critical threshold value for a heart frequency can be dependent on and/or be adapted from a resting pulse and a heart frequency in a calm phase of the patient 16. For example, a critical threshold value for the breathing signal of the patient 16 can be adapted to a breathing cycle in the resting state of the patient 16.

In an exemplary embodiment, if only a single state signal of the patient 16, in particular a state signal of just a single state category of the patient 16, is available to the evaluation algorithm for the establishment of the emotional state of the patient 16, then this state signal of the patient 16 must be greater than the predetermined critical threshold value. If on the other hand two of more different state signals, in particular different state signals from two or more different state categories, are available during an evaluation of state signals by the evaluation algorithm, then it can be defined by the evaluation algorithm that at least 50% of the two or more different state signals, in particular the different state signals from the two or more different state categories, must be greater than the respective critical threshold value for the determination of a critical emotional state of the patient 16.

In an exemplary embodiment, at least 55% of the different state signals, in particular of the different state signals from the two or more different state categories, fulfill the conditions for the determination of a critical emotional state of the patient 16. In an exemplary embodiment, at least 60% of the different state signals, in particular of the different state signals from the two or more different state categories, fulfill the conditions for the determination of a critical emotional state of the patient 16. In an exemplary embodiment, at least 65% of the different state signals, in particular of the different state signals from the two or more different state categories, fulfill the conditions for the determination of a critical emotional state of the patient 16. In an exemplary embodiment, at least 70% of the different state signals, in particular of the different state signals from the two or more different state categories, fulfill the conditions for the determination of a critical emotional state of the patient 16.

In an exemplary embodiment, in the second method step 101, for the evaluation algorithm, a trained artificial neural network can be employed for determining the emotional state of the patient 16. In particular the artificial neural network used for the evaluation is used to assign a detected facial expression of the patient 16 to an emotional state of the patient 16. In an exemplary embodiment, the artificial neural network has already been suitably trained beforehand for the evaluation of the detected state data. In this case training datasets are used in particular for the training of the artificial neural network, in which for example an emotional state is already assigned to a facial expression of a patient 16. The medical training datasets in this case have typically been acquired from training persons different from the patient 16 and/or from training patients. For example it is conceivable for the artificial neural network to take over the emotional state of the patient 16 established from the further detected state signals for the detected facial expression and/or the detected look on a patient's face. It is also conceivable for the detected facial expression and/or the detected look on the patient's face or information derived therefrom to be taken into consideration as additional training parameters during training of the artificial neural network.

In a third method step 102, in an exemplary embodiment, output information is generated based on the established emotional state of the patient 16 by the processor 27. The generated output information can include a text output. Moreover the output information can also include symbols that characterize the current established emotional state of the patient 16, such as for example symbols, which include an emoji and/or smiley.

Subsequently, in method step 102, the generated output information is provided by the processor 27 to be output and/or transferred to the output 25 for output. The output information can be output continuously to the user, in particular to the medical operating staff, by the output 25.

If, for example the patient is in a calm emotional state, this is communicated to the medical operating staff by the output 25 by showing appropriate output information, such as for example a happy smiley face and/or further output information. If on the other hand the patient 16 is in a nervous emotional state, this is communicated to the medical operating staff by the output 25 by showing appropriate output information, such as for example a sad smiley face and/or further output information.

Moreover, in such a case, when the patient 16 is in a nervous emotional state, a suggestion for calming down the patient 16 can be provided automatically by the processor 27 and communicated to the medical operating staff by the output 25. For example a suggestion for calming down the patient 16 can include changing an environment of the patient during the medical imaging examination, in particular the magnetic resonance examination. In particular in this case it can include a suggestion for adapting and/or changing illumination of the patient receiving area 15 and/or altering ventilation, in particular a flow of air within the patient receiving area 15, etc.

Moreover in such a case, when the patient 16 is in a nervous emotional state, a suggestion for adapting a measurement strategy and/or for adapting an examination strategy of the medical imaging examination, in particular of the magnetic resonance examination, on the basis of the established emotional state of the patient 16 can be determined and provided automatically by the processor 27. For example the suggestion provided by the processor 27 for an adaptation of the measurement strategy and/or of the examination strategy can include using fast measurement sequences and/or using quiet measurement sequences. Moreover the suggestion provided by the processor 27 for an adaptation of the measurement strategy and/or of the examination strategy can include a rearrangement of the measurement sequence for example, so that measurement sequences for acquiring the important image data are taken into account first. Moreover the suggestion provided by the processor 27 for an adaptation of the measurement strategy and/or of the examination strategy can include dispensing with an administration of contrast medium and/or additional examinations for example, so that the entire medical imaging examination, in particular the entire magnetic resonance examination, includes a measurement time that is as short as possible and thus the time that the patient 16 spends within the patient receiving area can be minimized and/or shortened.

In an exemplary embodiment, the suggestion for an adaptation of the measurement strategy and/or for an adaptation of the examination strategy of the medical imaging examination, in particular of the magnetic resonance examination is then provided by the processor 27 for output by the output 25 and communicated to the medical operating staff using the output 25. To accept such a suggestion, the medical operating staff merely have to confirm the suggestion, using the input 24, and the suggestion is then carried out automatically by the processor 25.

Although the disclosure has been illustrated and described in greater detail by the preferred exemplary embodiment, the disclosure is not restricted by the examples disclosed and other variations can be derived herefrom by the person skilled in the art, without departing from the scope of protection of the disclosure.

### CONCLUSION

The aforementioned description of the specific embodiments will so fully reveal the general nature of the disclosure that others can, by applying knowledge within the skill of the art, readily modify and/or adapt for various applications such specific embodiments, without undue experimentation, and without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general purpose computer.

For the purposes of this discussion, the term “processor circuitry” shall be understood to be circuit(s), processor(s), logic, or a combination thereof. A circuit includes an analog circuit, a digital circuit, state machine logic, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for monitoring a patient during a medical imaging examination, comprising:
detecting at least one state signal of the patient using at least one state detector;
evaluating, by a processor executing an evaluation algorithm, the at least one detected state signal of the patient, wherein the processor is configured to, using the evaluation algorithm, establish an emotional state of the patient based on the detected state signal; and
generating output information based on the established emotional state of the patient and outputting the output information via an output interface.

2. The method as claimed in claim 1, wherein the at least one detected state signal of the patient comprises: a signal of a movement of the patient, a signal of a detected facial expression of the patient, a signal of a heart cycle of the patient, a breathing signal of the patient, and/or a pulse signal of the patient.

3. The method as claimed in claim 1, wherein the detection of the at least one state signal of the patient comprises continuously detecting the at least one state signal of the patient during the medical imaging examination.

4. The method as claimed in claim 1, wherein at least two or more different state signals are detected and evaluated for the determination of the emotional state of the patient.

5. The method as claimed in claim 1, wherein the evaluation algorithm determines a change in the emotional state of the patient based on at least one change in the at least one detected state signal.

6. The method as claimed in claim 5, wherein the evaluation algorithm determines the change in the at least one detected state signal based on a comparison of the at least one detected state signal with a predetermined critical threshold value.

7. The method as claimed in claim 4, wherein the evaluation algorithm, for an evaluation of the at least two or more different state signals, is configured to determine a critical emotional state of the patient based on at least 50% of the at least two or more different state signals are greater than a corresponding predetermined critical threshold value.

8. The method as claimed in claim 1, wherein the evaluation algorithm is configured to employ a trained artificial neural network to determine the emotional state of the patient.

9. The method as claimed in claim 8, wherein the artificial neural network used for the evaluation assigns a detected facial expression to an emotional state of the patient.

10. The method as claimed in claim 1, wherein the output information is output continuously to a user.

11. The method as claimed in claim 1, wherein the processor is configured to automatically determine, based on the established emotional state of the patient, an adaptation of a measurement strategy and/or an examination strategy of the medical imaging examination.

12. A non-transitory computer-readable storage medium with an executable program stored thereon, wherein, when executed, the program instructs a processor to perform the method of claim 1.

13. A medical imaging apparatus adapted to monitor a patient during a medical imaging examination, comprising:
a scanner;
a patient receiving area surrounded by the scanner, the patient receiving area configured to receive the patient to be subjected to examination;
at least one state detector configured to detect at least one state signal of the patient;
a processor that is configured to execute an evaluation algorithm to:
evaluate the at least one detected state signal of the patient;
establish an emotional state of the patient based on the detected state signal;
generate output information based on the established emotional state of the patient; and
a user interface with an output interface that is configured to output the output information.

14. The medical imaging apparatus as claimed in claim 13, wherein the at least one state detector is integrated within the scanner.

15. The medical imaging apparatus as claimed in claim 13, wherein the at least one state detector comprises a camera and/or a breathing sensor and/or a pulse detector and/or a heart signal detector.

16. The medical imaging apparatus as claimed in claim 13, wherein the at least one detected state signal of the patient comprises: a signal of a movement of the patient, a signal of a detected facial expression of the patient, a signal of a heart cycle of the patient, a breathing signal of the patient, and/or a pulse signal of the patient.

17. The medical imaging apparatus as claimed in claim 13, wherein the processor is configured to determine a change in the emotional state of the patient based on at least one change in the at least one detected state signal.

18. The medical imaging apparatus as claimed in claim 17, wherein the processor is configured to compare the at least one detected state signal with a predetermined critical threshold value to determine the change in the at least one detected state signal.

19. The medical imaging apparatus as claimed in claim 13, wherein the medical imaging apparatus comprises two or more state detectors configured to detect two or more respective state signals of the patient, wherein the processor is configured to determine the emotional state of the patient based on at least 50% of the two or more respective state signals being greater than corresponding threshold values.

20. The medical imaging apparatus as claimed in claim 13, wherein the processor is configured to automatically adapt, based on the established emotional state of the patient, a measurement strategy and/or an examination strategy of the medical imaging examination.
